(19) United States
(12) Patent Application Publication
(10) Pub. No.: US 2006/0155350 A1
(43) Pub. Date: Jul. 13, 2006

(54) Pressure adjustable structure for ice compress

(76) Inventor: Nan Chih Lu, Tau Yuan Hsien (TW)

Correspondence Address: Nan Chih Lu, 235 Chung-Ho, Box 8-24, Taipei, TW

(21) Appl. No.: 11/031,141

(22) Filed: Jan. 10, 2005

(51) Int. Cl. A61F 7/00

(52) U.S. Cl. 607/114

(57) ABSTRACT

A pressure adjustable structure for ice compress compresses an ice-packing unit, an air-containing unit, and a pressure-adjusting unit, all included in an ice-compress body. The pressure-adjusting unit controls the gas pressure in the air-containing unit. By adding the air-containing unit and the pressure-adjusting unit to the conventional ice-packing unit, the pressure adjustable structure for ice compress acquires a function of arbitrarily adjusting the adhesive force of the ice-packing unit to a body portion it is attached to.

### FIELD OF THE INVENTION

The present invention relates to devices for ice compress, and more particularly to a pressure adjustable structure for ice compress capable of freely varying the gas pressure therein so that the effect of ice compress will be optimized.

### BACKGROUND OF THE INVENTION

Because of its simplicity, easy accessibility and effective medical functions, ice compress, as a means of cryotherapy, is a widely used medical treatment in daily life. Within the first 24 hours after an acute sport hazard, ice compress is used to alleviate pains, restrain inflammations, contract blood vessels, reduce edema and slow down metabolism, so as to cure the hazard.

A conventional means of ice compress uses a plastic bag filled with ice cubes, which will be applied onto a body portion. This method achieves the effect of ice compress on a small area, which is not an effective medical treatment. To achieve a better effect, the method of pressurized ice compress is adopted. It is done by binding the ice bag onto a body portion, such as an arm or a thigh, with a flexible binding strap. However, since the skin of the applied body portion cannot tolerate the low temperature of the ice bag immediately, the binding of the ice bag with the body portion should not be too tight. After a while, the adhesive stress should be increased to get better ice-compress effect.

Referring to FIG. 1, a structure for ice compress of the prior art comprises an ice-packing body 1 containing known a low-temperature preserving gel material. The ice-packing body 1 is frozen in advance in a refrigerator. The structure for ice compress mainly comprise a sheet body 10 having first adhesive sections 11 and a connection portion 120 in stalled on an exterior surface of the sheet body 10. Each of two lateral sides of the connection portion is extended with a flexible strap 12. The further side of each flexible strap 12 is provided with a second adhesive section 121. To use the structure for ice compress on a body portion, say a wrist portion 2, the sheet body 10 is wrapped around the wrist portion 2 and secured by the first adhesive sections 11, as shown in FIGS. 2 and 3. Secondly, each of the flexible straps 12 is stretched for a selected distance and pressed onto the outer surface of the sheet body 10, whereby the sheet body 10 will exert a pressure around the wrist portion 2. Finally, the flexible straps 12 are secured on the sheet body 10 by respective second adhesive sections 121, starting a continuous ice-compress effect, as shown in FIG. 4. To adjust the stress exerted on the wrist portion 2, the second adhesive sections 121 are firstly detached from the sheet body 10, and therefore the flexible straps 12 are restored to their original lengths. The above stretching process is then done again to get required binding stress. However, it is almost impossible to have a fine tuning of the binding stress, suing such a rough method.

Based on the disadvantages of the prior art, the inventor disclose the present invention for providing improvements.

### SUMMARY OF THE INVENTION

Accordingly, the primary objective of the present invention is to provide a pressure adjustable structure for ice compress, wherein fine adjustments of gas pressure therein can be done by freely inflating or deflating an air chamber therein. Thereby, the effect of ice compress can be optimized.

To achieve above object, the present invention provides a pressure adjustable structure for ice compress. The structure comprises an ice-packing unit being substantially a bag filled with a low-temperature preserving gel material; an air-containing unit disposed along tile ice-packing unit and substantially covering a surface of the ice-packing unit, the air-containing unit having a hollow air bag and a gas valve installed at a selected location; and a pressure-adjusting unit connected to the gas valve of the air-containing unit, the pressure-adjusting unit further comprising a gas tube, an air pumping unit, and a pressure-releasing unit, the gas tube having one end connected to the gas valve and another end connected to the air pumping unit through the pressure-releasing unit, whereby the air pumping unit will inject air into the air-containing unit and. The pressure-releasing unit will release excessive air in the air-containing unit to bring down the gas pressure therein.

The various objects and advantages of the present invention will be more readily understood from the following detailed description when read in conjunction with the appended drawings.

### BRIEF DESCRIPTION OF THE DRAWINGS

FIG. 1 is a perspective view of a structure for ice compress of the prior art.

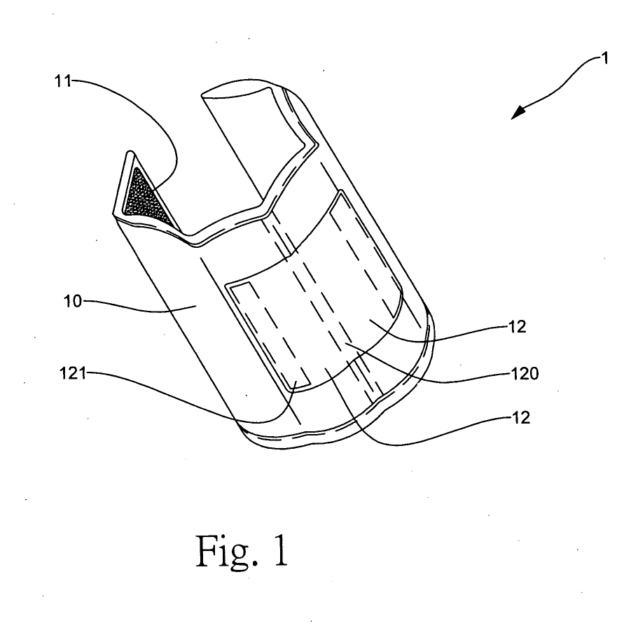

FIG. 2 illustrated the application of the structure for ice compress in FIG. 1 to a human body portion.

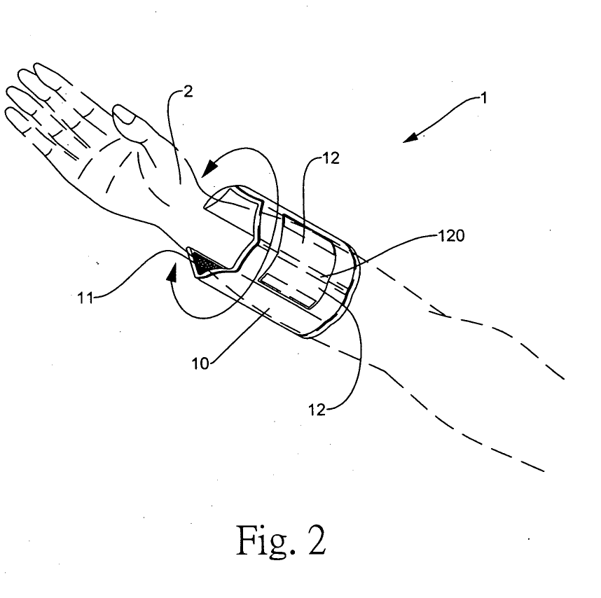

FIG. 3 is a second preferred embodiment of the structure for ice compress of the prior art.

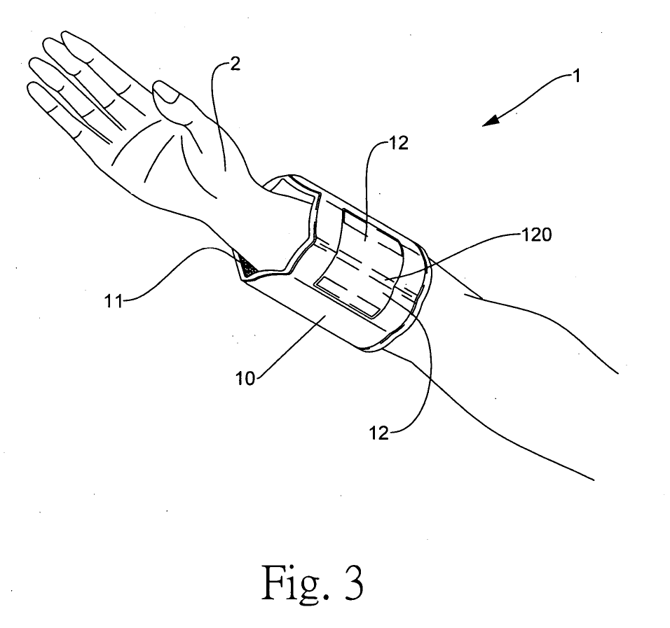

FIG. 4 is a third preferred embodiment of the structure for ice compress of the prior art.

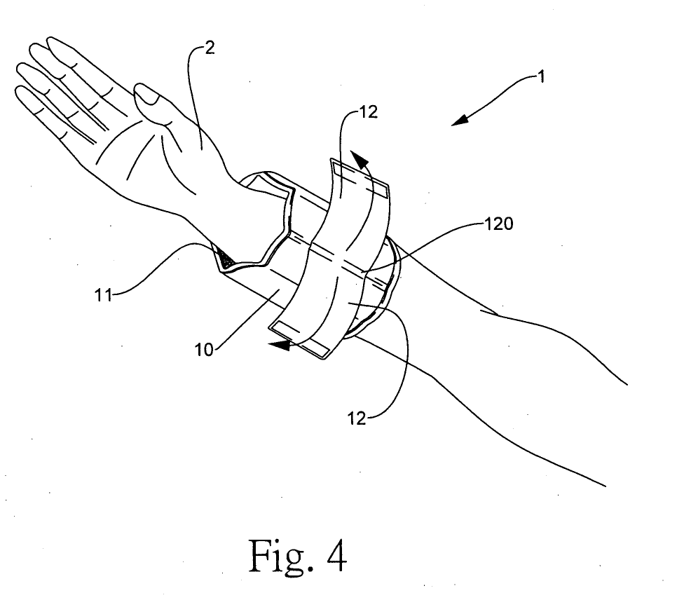

FIG. 5 is a perspective view of a pressure adjustable structure for ice compress according to the present invention.

FIG. 6 illustrated the application of the pressure adjustable structure for ice compress in FIG. 5 to a human body portion.

FIG. 7 is a top view of a second preferred embodiment of the present invention.

FIG. 8 is a cross-sectional view of the pressure adjustable structure for ice compress in FIG. 7.

FIG. 9 illustrated the application of the pressure adjustable structure for ice compress in FIG. 7 to a human body portion.

FIG. 10 is a perspective view of a third preferred embodiment of the present invention.

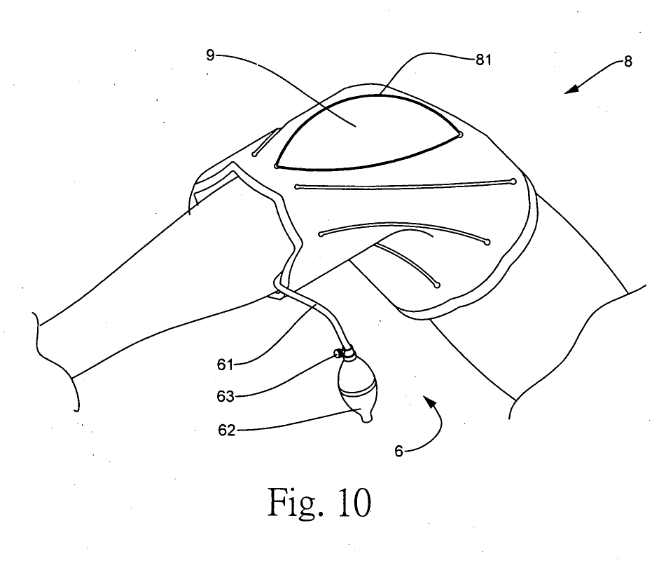

### DETAILED DESCRIPTION OF THE PREFERRED EMBODIMENTS

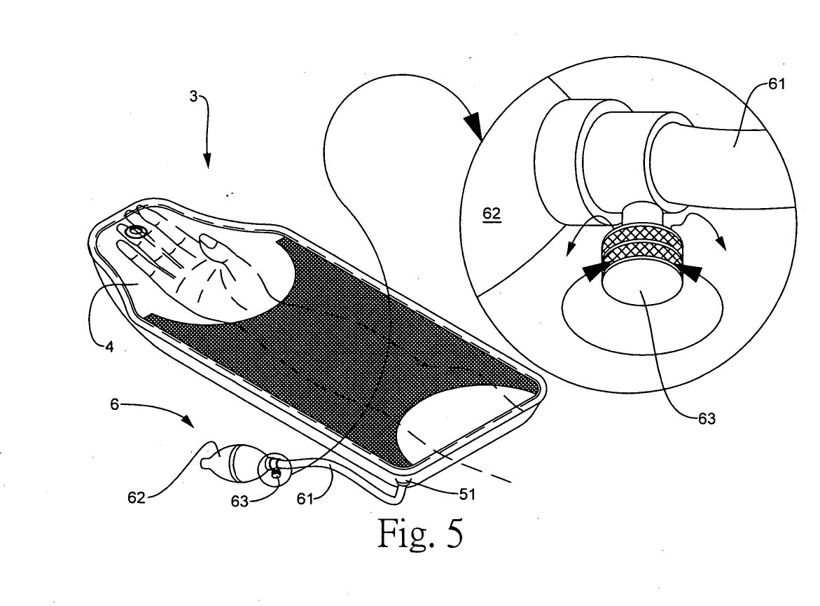

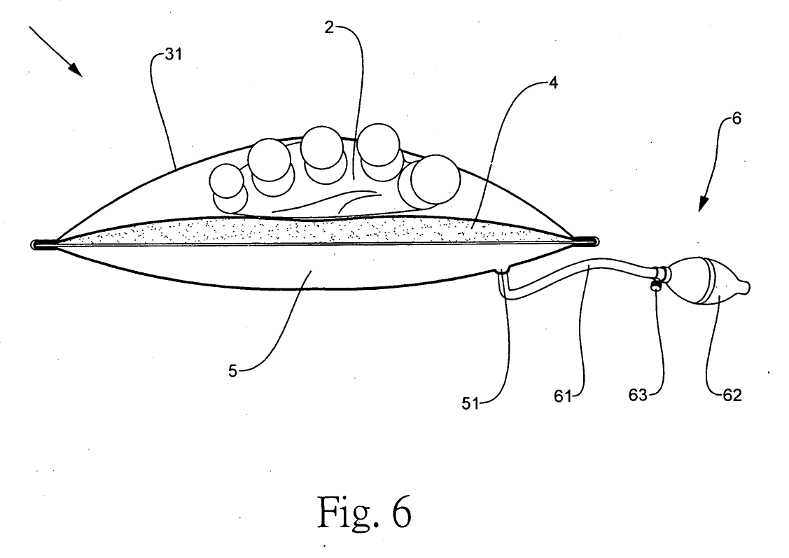

Referring to FIGS. 5 and 6, a pressure adjustable structure for ice compress according to the present invention comprises an ice-packing unit 4, an air-containing unit 5, and a pressure-adjusting unit 6, all included in an ice-compress body 3.

The ice-packing unit 4 is of bag structure, in which low-temperature preserving gel is stuffed. The gel is made of water, sodium, acidic groups, wood spirit, cellulose and pigments.

The air-containing unit 5 is a hollow air bag disposed along the ice-packing unit 4 and substantially covering the surface of the ice-packing unit 4. The air-containing unit 5 further includes a gas valve 51, which is connected to the pressure-adjusting unit 6.

The pressure-adjusting unit 6 comprises a gas tube 61, an air pumping unit 62, and a pressure-releasing unit 63. One end of the gas tube 61 is connected to the gas valve 51, and the other end is connected to the air pumping unit 62 through the pressure-releasing unit 63. As the air pumping unit 62 is squeezed, air is pumped into the air-containing unit 5 through the gas tube 61. As the pressure-releasing unit 63 is rotated or pressed, the air in the gas tube 61 will be released so as to bring the pressure in the air-containing unit 5 down.

The ice-compress body 3 further comprises a net body 31 for confining a hand between the net body 31 and the ice-packing unit 4, as shown in FIG. 6. The air pumping unit 62 is then squeezed and released repeatedly to inflate the air-containing unit 5. Thereby, the gas pressure in the air-containing unit 5 will push the ice-packing unit 4 against the hand to achieve an ice compress effect. As the temperature of the ice-packing unit 4 goes down, the gas pressure in the air-containing unit 5 can be brought up by further squeezing the air pumping unit 62. On the other hand, if the gas pressure is too high, the pressure-releasing unit 63 will be used to bring the pressure down.

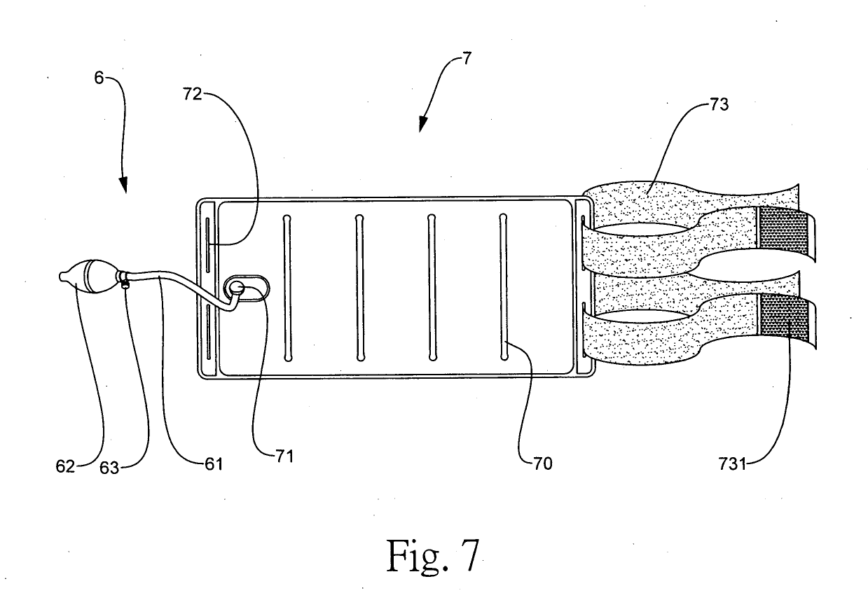

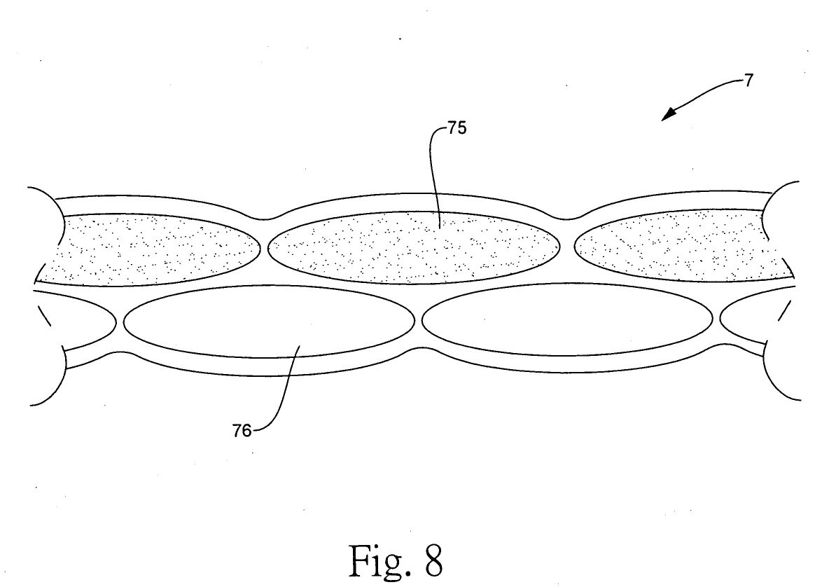

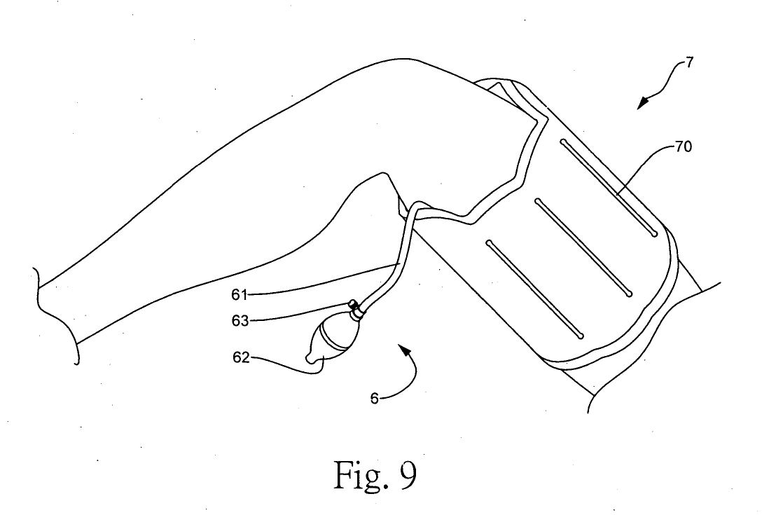

Another preferred embodiment of the present invention is shown in FIGS. 7 and 8, wherein an ice-compress body 7 comprises a plurality of ice-packing units 75, a plurality of air-containing units 76, and a pressure-adjusting unit 6. The ice-packing units 75 and the air-containing units 76 are further provided with a plurality of pressed dividers 70. The ice-compress body 7 further includes a gas valve 71 connected to the pressure-adjusting unit 6, a binding strap 73 with adhesive ends portions 731, and a plurality of insertion slots 72. To use this preferred embodiment, the ice-compress body 7 is brought to surround a body portion, such as a thigh portion, and secured by bringing the adhesive ends portions 731 of the binding strap 73 through the insertion slots 72 and engaging the adhesive ends portions 731 together, as shown in FIG. 9. The pressure-adjusting unit 6 is then squeezed to achieve the effect of pressured ice compress.

To facilitate the folding of the ice-compress body 7, the ice-packing units 75 and the air-containing units 76 are arranged in an interlaced fashion, as shown in FIG. 8.

In another preferred embodiment of the present invention, a ice-compress body 8 is provided with a hole 81, so that the pressurized ice compress can be applied around a knee 9.

In a larger sense, the present invention is applied not only to ice compress but also to hot compress.

The present invention is thus described, and it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A pressure adjustable structure for ice compress, comprising:
an ice-packing unit being substantially a bag filled with a low-temperature preserving gel material;
an air-containing unit disposed along said ice-packing unit and substantially covering a surface of said ice-packing unit, said air-containing unit having a hollow air bag and a gas valve installed at a selected location; and
a pressure-adjusting unit connected to said gas valve of said air-containing unit, said pressure-adjusting unit further comprising a gas tube, an air pumping unit, and a pressure-releasing unit, said gas tube having one end connected to said gas valve and another end connected to said air pumping unit through said pressure-releasing unit, whereby said air pumping unit will inject air into said air-containing unit and, and whereby said pressure-releasing unit will release excessive air in said air-containing unit to bring down the gas pressure therein.

2. The pressure adjustable structure for ice compress of claim 1 further comprising a net body for confining a body portion.

3. The pressure adjustable structure for ice compress of claim 1 wherein said ice-packing body further comprises a plurality of pressed dividers for facilitating folding said ice-packing body.

4. The pressure adjustable structure for ice compress of claim 1 wherein said ice-packing body is provided with at least one insertion slot and a binding strap with adhesive end sections, whereby said ice-packing body will be attached on a body portion by firstly having said strap go through said insertion slot and then securing said adhesive end sections together.

5. The pressure adjustable structure for ice compress of claim 1 wherein said ice-packing body is provided with a hole for accommodating a joint portion of a animal body.

6. The pressure adjustable structure for ice compress of claim 1 being further applicable to hot compress.
